SEQUENCE LISTING (2 sequences)

SEQ ID NO: 1 - protein, 34 aa, Homo sapiens
AVSEHQLLHDKGKSIQDLRRRFFLHHLIAEIHTA

SEQ ID NO: 2 - protein, 34 aa, Artificial Sequence
  Synthetic Peptide
  VARIANT (29)..(29): Xaa = 2-aminoisobutyric acid
  MOD_RES (34)..(34): AMIDATION
AVSEHQLLHDKGKSIQDLRRRELLEKLLXKLHTA

(19) United States
(12) Patent Application Publication
(10) Pub. No.: US 2018/0161401 A1
(43) Pub. Date: Jun. 14, 2018

(54) Novel Formulations of PTHrP Analogue

(71) Applicant: Zheng Xin Dong, Holliston, MA (US)

(72) Inventor: Zheng Xin Dong, Holliston, MA (US)

(21) Appl. No.: 15/579,589

(22) PCT Filed: Jul. 5, 2016
(86) PCT No.: PCT/US16/41016

§ 371 Date: Dec. 4, 2017

Related U.S. Application Data
(60) Provisional application No. 62/189,162, filed on Jul. 6, 2015.
(60) Provisional application No. 62/357,358, filed on Jun. 30, 2016.

(51) Int. Cl. A61K 38/29; A61K 47/10; A61K 9/00; A61P 19/10

(52) U.S. Cl. CPC A61K 38/29; A61P 19/10; A61K 9/0019; A61K 47/10

(57) ABSTRACT

The present invention provides a non-buffered, neutral pH easily prepared, storage-stable composition containing a parathyroid hormone-related protein (PTHrP) analogue and methods of using a PTHrP analogue and the PTHrP compositions described herein to treat osteoporosis, to increase bone mass or to increase bone quality. The composition has a non-buffered neutral pH which avoids injection site reactions and is easy to prepare and storage stable, in sterile form, and in general may be stored at room temperature for at least several weeks to allow convenient parenteral administration to human patients.

### BACKGROUND OF THE INVENTION

Parathyroid hormone-related protein (“PTHrP”) is a 139 to 173 amino acid-protein. PTHrP and certain analogs are known to be useful to improve bone mass and quality in the treatment of osteoporosis and related disorders. However, the commercial use of these proteins as pharmaceutical agents requires the development of a formulation that is acceptable in terms of storage stability, ease of preparation and suitable for subcutaneous injections without inducing injection site reactions such as irritation to an acidic solution with a buffered acidic pH.

Furthermore, currently available osteoporosis drugs have limitations on suitable dosage ranges due to the unwanted side-effects, such as hypercalcemia and increased stimulation of bone resorption. These unwanted side-effects and resulting dose limitations reduce the beneficial effects which can be achieved from these drugs. Thus, a need exists for compounds which can be administered at a dose which will increase the beneficial effects without an increase in the unwanted side-effects.

### SUMMARY OF THE INVENTION

The present invention provides a storage-stable, ease-to-prepare composition containing a parathyroid hormone-related protein (PTHrP) analogue and methods of using the analogue and compositions containing the analogue as described herein to treat osteoporosis, to increase bone mass or to increase bone quality. The composition is storage stable, easy to prepare, in sterile form, suitable for subcutaneous injections without inducing injection site reactions such as the reactions to an acid solution with a buffered acidic pH, and in general may be stored at room temperature for at least several weeks to allow convenient parenteral administration to human patients.

In one embodiment, the present invention provides a storage-stable, easily prepared composition suitable for subcutaneous administration to a subject (e.g., a human) at pH close to that of the physiological condition. The composition comprises a PTHrP analogue without a chemical buffer, which ensures that after subcutaneous injection the composition is rapidly neutralized to the physiological pH without inducing any injection site irritation. In a particular embodiment, the PTHrP is [Glu^{22,25}, Leu^{23,28,31}, Aib²⁹, Lys^{26,30}]hPTHrP (1-34)NH₂ SEQ ID NO.: 2). The previously reported formulation of [Glu^{22,25}, Leu^{23,28,31}, Aib²⁹, Lys^{26,30}]hPTHrP (1-34)NH₂ (SEQ ID NO.: 2) used acidic pH (e.g., pH 5.1) that is much lower than physiological pH 7.4, which was further buffered with acetate to maintain the acidic pH after injection. Therefore, such a buffered acidic solution cannot be rapidly neutralized by the body fluid at the injection site, resulting in injection site reactions (Leder B Z et al., J Clin Endocrin Metab, 2015, 100 (2):697-706). Forteo® (Teriparatide, 0.6 mg/2.4 ml, subcutaneous injection) (Eli Lilly & Co., Indianapolis, Ind.), a parathyroid hormone (PTH) used for the treatment of osteoporosis, is also formulated in an acidic solution with a pH at 4 and buffered with acetate. Similarly, Foreteo® also causes injection site reactions including injection site pain, swelling and bruising (www.forteo.com). In contrast to these buffered acidic PTHrP and PTH formulations, the invention herein uses the formulations with the pH close to the physiological pH 7.4 and without any buffer system. These formulations are rapidly neutralized so the physiological pH at the injection site and therefore minimize the injection site reactions. The invention also includes the use of formulations with buffered physiological pH of 7.4. Because of its physiological pH, the formulations minimize injection site reactions.

In another embodiment, the present invention provides a sealed container containing a storage-stable composition suitable for administration to a subject. The composition comprises PTHrP or an analog thereof and an effective amount of buffer to maintain the pH of the composition between 6.0 and 8.5, which is close to the physiological pH, to avoid injection site irritation and reactions. In a particular embodiment, the PTHrP analogue is [Glu^{22,25}, Leu^{23,28,31}, Aib²⁹, Lys^{26,30}]hPTHrP(1-34)NH₂ (SEQ ID NO.:2).

In another embodiment, the present invention provides a drug delivery device comprising one or more than one single-use container which comprises a storage stable composition comprising PTHrP or an analog thereof without any chemical buffer to avoid buffered acidic solution-induced injection site reactions. In a particular embodiment, the PTHrP analogue is [Glu^{22,25}, Leu^{23,28,31}, Aib²⁹, Lys^{26,30}]hPTHrP(1-34)NH₂ (SEQ ID NO.:2).

In another embodiment, the present invention provides a drug delivery device comprising one or more than one multi-use container, which comprises a storage stable, easily prepared composition comprising PTHrP or an analog thereof and an effective amount of butter to maintain the pH of the composition close to neutral pH to avoid the injection site reactions. In a particular embodiment, the PTHrP analogue is [Glu^{22,25}, Leu^{23,28,31}, Aib²⁹, Lys^{26,30}]hPTHrP(1-34)NH₂ (SEQ ID NO.: 2).

In another embodiment the present invention provides a method of treating osteoporosis in a subject in need thereof comprising administering to the subject a single daily subcutaneous dose [Glu^{22,25}, Leu^{23,28,31}, Aib²⁹, Lys^{26,30}]hPTHrP (1-34)NH₂ (SEQ ID NO.: 2) in an amount between 70 and 120 μg for a duration of time sufficient to treat the subject, typically between about 3 months to 36 months. In some embodiments, the treatment period is between about 3 months to 18 months.

In another embodiment, the present invention provides a method of increasing bone mass or increasing hone quality in a subject in need thereof comprising administering to the subject a single daily subcutaneous dose of [Glu^{22,25}, Leu^{23,28,31}, Aib²⁹, Lys^{26,30}]hPTHrP (1-34)NH₂ (SEQ ID NO. 2) in an amount between 70 and 120 μg for a duration of time sufficient to treat the subject, typically between 3 months and 36 months. In some embodiments, the treatment period is between about 3 months to 18 months.

The PTHrP and analogue compositions of the invention exhibit storage stability in terms of hormone composition and activity and ease of preparation. Furthermore, these compositions can be administered, in general, at higher dosages than currently available osteoporosis drugs, with the reduction or elimination of unwanted side-effects, such as injection site reactions, hypercalcemia or stimulation of bone resorption. This has the advantage of an increase in beneficial physiological effects due to the increased dosages and can result in a reduction in the length of treatment time.

### DETAILED DESCRIPTION OF THE INVENTION

The sequence of native hPTHrP (1-34) is as follows:

(SEQ ID NO: 1)
Ala Val Ser Glu His Gln Leu Leu His Asp Lys Gly
Lys Ser Ile Gln Asp Leu Arg Arg Arg Phe Phe Leu
His His Leu Ile Ala Glu Ile His Thr Ala.

In a particular embodiment, the PTHrP analogue is [Glu^{22,25}, Leu^{23,28,31}, Aib²⁹, Lys^{26,30}]hPTHrP(1-34)NH₂, which is Ala Val Ser Glu His Gln Leu Leu His Asp Lys Gly Lys Ser Ile Gln Asp Leu Arg Arg Arg Glu Leu Leu Glu Lys Leu Leu Aib Lys Leu His Thr Ala-NH₂ (SEQ ID NO.: 2).

Other PTHrP analogues are described in U.S. Pat. Nos. 6,921,750, 5,355,574, 6,544,949, 5,723,577, and 5,696,095 the entire contents of each of which are incorporated herein by reference.

A “buffer,” as used herein, is any acid or salt combination which is pharmaceutically acceptable and capable of maintaining the composition of the present invention within a desired pH range. Buffers, in the disclosed compositions, maintain the pH in a range of about 2 to about 8.5 about 5.0 to about 8.0 about 6.0 to about 7.5, about 6.5 to about 7.5, or about 6.5. Suitable buffers include, any pharmaceuticals acceptable buffer capable of maintaining the above pH ranges, such as, for example, acetate, tartrate, phosphate, succinate, maleate, imidazole or citrate buffers. In one embodiment, the buffer is an acetate or phosphate buffer. In another embodiment, the buffer is an acetate buffer. In a further embodiment, the buffer is acetic acid and sodium acetate. In another embodiment the buffer is a phosphate buffer, such as phosphate-buffered saline (PBS). In yet another embodiment, the buffer is disodium phosphate and monosodium phosphate.

For the disclosed compositions, the concentration of buffer is typically in the range of about 0.1 mM to about 1000 mM, about 0.2 mM to about 200 mM, about 0.5 mM to about 50 mM, about 1 mM to about 10 mM or about 6 mM.

As used herein, an anti-microbial agent is a pharmaceutically acceptable preservative, suitable for administration to a subject, which inhibits, prevents or delays the growth or microorganisms including, for example bacteria, viruses and fungi in the compositions of the present invention. Suitable anti-microbial agents for use in the compositions and methods of the present invention include, but are not limited to, cresols, benzyl alcohol, phenol, benzalkonium chloride, benzethonium chloride, chlorobutanol, phenylethyl alcohol, methyl paraben, propyl paraben, thiomersal and phenylmercuric nitrate and acetate. In one embodiment, the anti-microbial agent is m-cresol, chlorocresol or phenol. In another embodiment, the anti-microbial agents is chlorocresol or phenol. In another embodiment the anti-microbial agent is phenol.

As used herein, an “effective amount” of an anti-microbial agent is an amount effective to inhibit, prevent or delay the growth or microorganisms including, for example bacteria, viruses and fungi, in the compositions of the present invention. In the compositions of the present invention, the amount of anti-microbial agent is typically in the range from about 0.1 to about 20 mg/ml, about 0.2 to about 30 mg/ml, about 0.2 to about 50 mg/ml about 0.25 to about 5 mg/ml, about 0.5 to about 50 mg/ml, about 1 to about 10 mg/ml, about 3 mg/ml or about 5 mg/ml.

The term “about” as used herein is defined as “±5%”.

The compositions of the present invention typically are ready to administer, aqueous solutions, which are sterile, storage-stable and pharmaceutically acceptable without the need for reconstitution prior to administration. The compositions of the present invention are suitable for administration to a subject which means that they are pharmaceutically acceptable, non-toxic, do not contain any components which would adversely affect the biological or hormonal effects of the peptide, and have the pH close to that of the physiological condition which avoids injection site reactions. The compositions of the present invention do not, for example, comprise any cells.

The compositions are typically stored in a sealed container, vial or cartridge which is typically suitable for long term storage. “Suitable for long-term storage” means that the vial, container or cartridge does not allow for the escape of components of the compositions of the present invention or the ingress of external components, such as, microorganisms when kept for at least 3 months at 25° C.

The compositions of the present invention are preferably administered by injection, typically subcutaneous injection.

The compositions of the present invention, can be stored in single-dose or multi-dose sealed containers, vials or cartridges. The sealed container, vial or cartridge is typically suitable for use with a single or multi-dose injection pen or drug delivery device, which typically allows the patient to administer the peptide themselves. The sealed container can comprise one or more doses of the peptide of the present invention, wherein each dose comprises an effective amount of the peptide as described herein.

A single-dose injection pen, or drug delivery device is typically a disposable device which uses a sealed container which comprises a single dose of an effective amount of a PTHrP in the compositions described herein. A multi-dose injection pen or drug delivery device typically contains more than one dose of an effective amount of a PTHrP thereof in the compositions described herein. The multi-dose pen can typically be adjusted to administer the desired volume of the storage stable compositions described herein. In certain embodiments, the multi-dose injection pen prevents the ingress of microbial contaminants into the container or cartridge which can occur through multiple uses of one needle.

Injection pens, as used herein, can also comprise two containers one of which contains a PTHrP, as described herein, in a lyophilized powder, as described below, and a second container that contains a liquid for reconstitution of the lyophilized powder. The contents of the two containers can be mixed prior to administration.

As discussed above, the compositions of the present invention can be administered by injection. Suitable volumes of the compositions of the present invention for injection include about 0.5 to about 1 ml, about 0.1 to about 1 ml, about 0.02 to about 0.04 ml, about 0.1 to about 5.0 μl, and about 0.1 to about 1 μl.

In the compositions of the present invention, the concentration of the peptides is from about 0.1 mg/ml to about 10.0 mg/ml, from about 10.0 mg/ml to about 100.0 mg/ml, from about 30.0 mg/ml to about 300.0 mg/ml, from about 500 mg/ml to about 2000 mg/ml and about 2.0 mg/ml.

The compositions of the present invention can also be lyophilized using lyophilization techniques known in the art and stored as a powder which can be reconstituted prior to administration. The term “lyophilization,” as used herein, is a freeze drying or dehydration technique which involves removing a solvent, preferably a water miscible solvent, more preferably water from a composition or the present invention, typically by sublimation under high vacuum when the composition is in a frozen state. Typically, lyophilization is carried out in lyophilization equipment (a lyophilizer), which comprises a drying chamber with variable temperature controls, a condenser to collect water, and a vacuum system to reduce the pressure in the drying chamber.

The term “lyophilized composition,” as used herein, indicates that a solid residue or powder was produced by the lyophilization procedure defined above. A lyophilized composition of the present invention typically further comprises a pharmaceutically acceptable excipient. The term “pharmaceutically acceptable excipient,” as used herein, refers to a substance which is added to a solution prior to lyophilization to enhance characteristics such as the color, texture, strength, and volume of the lyophilized cake. Pharmaceutically acceptable excipients include, for example, buffers and pH adjusters, crystalline bulking excipients, stabilizers, and tonicity raising agents.

In certain preferred embodiments, the pharmaceutically acceptable excipient is a crystalline bulking excipient. The terms “crystalline bulking excipient” or “crystalline bulking agent,” as used herein, refer to an excipient which provides bulk and structure to the lyophilization cake. These crystalline bulking agents are inert and do not react with the peptide. In addition, the crystalline bulking agents are capable of crystallizing under lyophilization conditions.

Examples of suitable crystalline bulking agents include hydrophilic excipients, such as, water soluble polymers; sugars, such as mannitol, sorbitol, xylitol, glucitol, ducitol, inositiol, arabinitol, arabitol, galactitol, iditol, allitol, maltitol, fructose, sorbose, glucose, xylose, trehalose, allose, dextrose, altrose, lactose, glucose, fructose, gulose, idose, galactose, talose, ribose, arabinose, xylose, lyxose, sucrose, maltose, lactose, lactulose, fucose, rhamnose, melezitose, maltotriose, raffinose, altritol, their optically active forms (D- or L-forms) as well as the corresponding racemates; inorganic salts, both mineral and mineral organic, such as, calcium salts, such as the lactate, gluconate, glycerylphosphate, citrate, phosphate monobasic and dibasic, succinate, sulfate and tartrate, as well as the same salts of aluminum and magnesium; carbohydrates, such as, the conventional mono- and di-saccharides as well as the corresponding polyhydric alcohols; proteins, such as, albumin; amino acids, such as glycine; emulsifiable fats and polyvinylpyrrolidone. Preferred crystalline bulking agents are selected from the group consisting of glycine, mannitol, dextran, dextrose, lactose, sucrose, polyvinylpyrrolidone, trehalose, glucose and combinations thereof. A particularly useful bulking agent is dextran.

As used herein, a “stabilizer” is a composition which maintains the chemical, biological or hormonal stability of the peptide. Examples of stabilizing agents include polyols, such as, for example, saccharide, preferably a monosaccharide or disaccharide, e.g., glucose, trehalose, raffinose, or sucrose; a sugar alcohol, such as, for example, mannitol, sorbitol or inositol; a polyhydric alcohol, such as, for example, glycerine or propylene glycol; or mixtures thereof, and albumin.

The compositions described herein can be used to stimulate bone growth in a subject and are, therefore, useful in the treatment of diseases or disorders associated with deficiency in bone growth, such as osteoporosis and bone fractures. In one embodiment, the present invention is directed to a method of treating osteoporosis in a subject comprising administering to the subject an effective amount of composition described herein.

As used herein, “treating” can include both prophylactic and therapeutic treatment. For example, therapeutic treatment can include delaying, inhibiting or preventing the progression of osteoporosis, and/or reducing or eliminating the symptoms associated with osteoporosis. Prophylactic treatment can include preventing, inhibiting or delaying the onset of osteoporosis.

As used herein, an “effective amount” refers to an amount sufficient to elicit the desired response. In the present invention, the desired biological response is a decrease in the rate of bone loss and/or an increase in the bone mass or bone quality of a subject.

Suitable dosages of the present invention include from about 40 to about 160 μg, about 70 to about 120 μg, and about 80 to about 100 μg, administered once per day, once every other day, twice per week, once per week, once every two weeks or once per month. The doses can be a pulsatile injection, for example, once per month which causes pulsatile release of singles doses of the composition described herein.

“The subject,” as used herein, can be an animal, for example, a mammal, such as a human.

A “pharmaceutically acceptable salt” is a salt which is suitable for administration to a subject, such as a human. The peptides of the present invention can have one or more sufficiently acidic protons that can react with a suitable organic or inorganic base to form a base addition salt. Base addition salts include those derived from inorganic bases, such as ammonium or alkali or alkaline earth metal hydroxides, carbonates, bicarbonates, and the like, and organic bases such as alkoxides, alkyl amides, alkyl and aryl amines, and the like. Such bases are useful in the preparation of the salts of this invention and include, for example, sodium hydroxide, potassium hydroxide, ammonium hydroxide, potassium carbonate, and the like. Peptides of the present invention having a sufficiently basic group, such as an amine, can react with an organic or inorganic acid to form an acid addition salt. Acids commonly employed to form acid addition suits from compounds with basic groups are inorganic acids, such as hydrochloric acid. hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include sulfate, pyrosulfate, bisulfate, sulfite, bisulfite, phosphate, monohydrogenphosphate, dihydrogenphosphate, etaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

The compositions of the present invention typically do not show any, or show reduced, side-effects such as hypercalcemia, typically do not increase the stimulation of bone resorption at the dosage listed above, and do not induce injection site reactions such as irritation due to their neutral pH and/or non-buffered solutions. This reduction in side effects allows for administration of higher doses than commercially available osteoporosis drugs.

The compositions of the present invention can be administered by injection as described herein.

The compositions of the present invention may be administered alone, or in combination with, an additional therapeutic agent, such as an antiresorptive therapy, for example, bisphonsphonates and calcitonin.

It will be understood by those skilled in the art that various changes in form and details may be made therein to the compositions without departing from the scope of the invention encompassed by the appended claims.

### Exemplification

### EXAMPLE 1

[Glu^{22,25}, Leu^{23,28,31}, Aib²⁹, Lys^{26,30} ]hPTHrP(1-34)NH₂ (SEQ ID NO.:2) Formulation With pH 6.5 Without a Chemical Buffer

[Glu^{22,25}, Leu^{23,28,31}, Aib²⁹, Lys^{26,30}]hPTHrP(1-34)NH₂ (SEQ ID NO.:2) 5.0 mg (free base) was dissolved in 4.50 mL of Water for Injection. The pH of the resulting solution was adjusted to 6.5 by using 1% hydrochloric acid solution and 1% sodium hydroxide solution and final volume of the solution was adjusted to 5.0 mL with Water for Injection.

### EXAMPLE 2

### [Glu^{22,25}, Leu^{23,28,31}, Aib²⁹, Lys^{26,30}]hPTHrP(1-34)NH₂ (SEP ID NO.:2) Formulation With pH 7.5 in Phosphate Buffer

[Glu^{22,25}, Leu^{23,28,31}, Aib²⁹, Lys^{26,30}]hPTHrP(1-34)NH₂ (SEQ ID NO.:2) 5.0 mg (free base) was dissolved in 5.0 mL of pH 7.5 phosphate buffer (10 mM, Na₂HPO₄ and NaH₂PO₄). The pH of the resulting solution was 7.5.

### EXAMPLE 3

[Glu^{22,25}, Leu^{23,28,31}, Aib²⁹, Lys^{26,30}]hPTHrP(1-34)NH₂ (SEQ ID NO.:2) Formulation With pH 6.5 in the Presence of Phenol at the Concentration of 5.6 mg/mL and Without Chemical Buffer

[Glu^{22,25}, Leu^{23,28,31}, Aib²⁹, Lys^{26,30}]hPTHrP(1-34)NH₂ (SEQ ID NO.:2) 5.0 mg (free base) was dissolved in 4.60 mL of Water for Injection containing 5.0 mg/mL of phenol. The pH of the resulting solution was adjusted to 6.5 by using 1% hydrochloric acid solution and 1% sodium hydroxide solution and final volume of the solution was adjusted to 5.0 mL with Water for Injection containing phenol at the concentration of 5.0 mg/mL.

### EXAMPLE 4

### [Glu^{22,25}, Leu^{23,28,31}, Aib²⁹, Lys^{26,30}]hPTHrP(1-34)NH₂ (SEQ ID NO.:2) at pH 7.5 With Phosphate Buffer Containing Phenol at the Concentration of 5.0 mg/mL

[Glu^{22,25}, Leu^{23,28,31}, Aib²⁹, Lys^{26,30}]hPTHrP(1-34)NH₂ (SEQ ID NO.:2) 5.0 mg (free base) was dissolved in 5.0 mL of pH 7.5 phosphate buffer (Na₂HPO₄ and NaH₂PO₄, 10 mM) containing phenol at the concentration of 5.0 mg/mL phenol. The pH of the resulting solution was 7.5.

### EXAMPLE 5

### [Glu^{22,25}, Leu^{23,28,31}, Aib²⁹, Lys^{26,30}]hPTHrP(1-34)NH₂ (SEQ ID NO.:2) Formulation With pH 6.5 in the Presence of Phenol at the Concentration of 5.0 mg/mL and Mannitol at the Concentration of 18 mg/mL, Without Chemical Buffer

[Glu^{22,25}, Leu^{23,28,31}, Aib²⁹, Lys^{26,30}]hPTHrP(1-34)NH₂ (SEQ ID NO.:2) 2.0 mg (free base) was dissolved in 0.80 mL of Water for Injection containing 5.0 mg/mL of phenol and 18 mg/mL of mannitol. The pH of the resulting solution was adjusted to 6.5 by using 1% hydrochloric acid solution and 1% sodium hydroxide solution and final volume of the solution was adjusted to 2.0 mL with Water for Injection containing phenol at the concentration of 5.0 mg/mL and mannitol at the concentration of 18 mg/mL. The pH of the resulting solution was 6.5.

### EXAMPLE 6

### [Glu^{22,25}, Leu^{23,28,31}, Aib²⁹, Lys^{26,30}]hPTHrP(1-34)NH₂ (SEQ ID NO.:2) at pH7.5 With Phosphate Buffer (Na₂HPO₄ and NaH₂PO₄, 10 mM) Containing Phenol at the Concentration of 5.0 mg/mL and Mannitol at the Concentration of 18 mg/mL

[Glu^{22,25}, Leu^{23,28,31}, Aib²⁹, Lys^{26,30}]hPTHrP(1-34)NH₂ (SEQ ID NO.:2) 2.0 mg (free base) was dissolved in 2.0 mL of pH 7.5 phosphate buffer (Na₂HPO₄ and NaH₂PO₄, 10 mM) containing phenol at the concentration of 5.0 mg/mL and mannitol at the concentration of 18 mg/mL. The pH of the resulting solution was 7.5.

### EXAMPLE 7

### [Glu^{22,25}, Leu^{23,28,31}, Aib²⁹, Lys^{26,30}]hPTHrP(1-34)NH₂ (SEQ ID NO.:2) Formulation With pH7.5 in the Presence of Phenol at the Concentration of 5.0 mg/mL and Mannitol at the Concentration of 18 mg/mL, Without Chemical Buffer

[Glu^{22,25}, Leu^{23,28,31}, Aib²⁹, Lys^{26,30}]hPTHrP(1-34)NH₂ (SEQ ID NO.:2) 4.0 mg (free base) was dissolved in 3.6 mL of Water for Injection containing 5.0 mg/mL of phenol and 18 mg/mL of mannitol. The pH of the resulting solution was adjusted to 7.5 by using 1% hydrochloric acid solution and 1% sodium hydroxide solution and final volume of the solution was adjusted to 5.0 mL with Water for Injection containing phenol at the concentration of 5.0 mg/mL and 18 mg/mL of mannitol. The pH of the resulting solution was 7.5.

TABLE I
Formulations of [Glu^{22,25}, Leu^{23,28,31},
Aib²⁹, Lys^{26,30}]hPTHrP(1-34)NH₂ (SEQ ID NO.: 2)

| | Formulation of [Glu^{22,25}, Leu^{23,28,31}, Aib²⁹, | | | | |
|---|---|---|---|---|---|
| | Lys^{26,30}]hPTHrP(1-34)NH₂ (SEQ ID NO.: 2) | | | | |
| | | | Concen- | Phenol | Mannitol |
| | | | tration | concen- | concen- |
| | | | of peptide | tration | tration |
| | pH | Buffer | (mg/mL) | (mg/mL) | (mg/mL) |
| Example 1 | 6.5 | None | 1.0 | None | None |
| Example 2 | 7.5 | Phosphate | 1.0 | None | None |
| Example 3 | 6.5 | None | 1.0 | 5.0 | None |
| Example 4 | 7.5 | Phosphate | 1.0 | 5.0 | None |
| Example 5 | 6.5 | None | 1.0 | 5.0 | 18 |
| Example 6 | 7.5 | Phosphate | 1.0 | 5.0 | 18 |
| Example 7 | 7.5 | None | 1.0 | 5.0 | 18 |

### EXAMPLE 8

### Injection Site Reaction Studies in Athymic Nude Mice

Eight groups of athymic nude mice (Charles River Laboratories International. Inc., Wilmington, Mass.), 3 animals per group, were subcutaneously (s.c.) administered with the formulations of [Glu^{22,25}, Leu^{23,28,31}, Aib²⁹, Lys^{26,30}]hPTHrP(1-34)NH₂ (SEQ ID NO.:2) in Examples 1, 2, 3, 4, 5, 6 and 7, respectively. The injection schedule for each animal group was once daily for 5 consecutive days. Syringes with 33 gauge needles were used for the injections. After each injection, the injection site was visually inspected to identify any injection site reactions such as redness, swelling and/or bruising. No injection site reactions were observed for any of these formulations tested (Table II).

### EXAMPLE 9

### Infection Site Reaction Studies in Rabbits

Groups of New Zealand Albino rabbits (Charles River Laboratories International, Inc., Wilmington, Mass.), 2 animals per group, were subcutaneously (s.c.) administered with the formulations of [Glu^{22,25}, Leu^{23,28,31}, Aib²⁹, Lys^{26,30}]hPTHrP(1-34)NH₂ (SEQ ID NO.:2) in Examples 1, 2, 3, 4, 5, 6 and 7, respectively. The injection schedule for each animal group was once daily for 5 consecutive days. The injection volume was 100 μL per injection per animal. Syringes with 30 gauge needles were used for the injections. After each injection, the injection site was visually inspected to identify any injection site reactions such as redness, swelling or bruising. No injection site reactions were observed for any of these formulations tested.

TABLE II
Injection site reaction study*

| | Formulation of [Glu^{22,25}, Leu^{23,28,31}, Aib²⁹, | | |
|---|---|---|---|
| | Lys^{26,30}]hPTHrP(1-34)NH₂ (SEQ ID NO.: 2) | | |
| | | | Subcutaneous injection |
| | Injection | Treatment | site reactions (e.g., |
| | volume | period | redness, swelling |
| | (μL) | (days) | and/or bruising) |
| Example 1 | 20 | 5 | None |
| Example 2 | 20 | 5 | None |
| Example 3 | 20 | 5 | None |
| Example 4 | 20 | 5 | None |
| Example 5 | 20 | 5 | None |
| Example 6 | 20 | 5 | None |
| Example 7 | 20 | 5 | None |

*Athymic nude mice (Charles River Laboratories International, Inc., Wilmington, Massachusetts), 3 animals per group, were subcutaneously administered with the formulations of [Glu^{22,25}, Leu^{23,28,31}, Aib²⁹, Lys^{26,30}]hPTHrP(1-34)NH₂ (SEQ ID NO.: 2) in Examples 1 to 8, respectively, once daily for 5 days.

1. A composition suitable for administration to a subject comprising a PTHrP analogue having the sequence [Glu^{22,25}, Leu^{23,28,31}, Aib²⁹, Lys^{26,30}]hPTHrP(1-34)NH₂ (SEQ ID NO.2),
wherein the pH of the composition is in a range of about 3.5 to about 8.5;
provided that wherein said composition does not contain an effective amount of a pH buffer for maintaining the pH from about 6.0 to about 8.0.

2. (canceled)

3. A composition according to claim 1, wherein said pH is about 6.5.

4. A composition according to claim 1, further comprising an effective amount of an antimicrobial agent, wherein said antimicrobial agent maintains the stability of said composition for storage prior to use.

5. A storage-stable composition according to claim 4, wherein said anti-microbial agent is phenol.

6. A composition according to claim 5, wherein said phenol is present in a concentration from about 0.25 mg/mL to about 7 mg/mL.

7. A composition according to claim 5, wherein said phenol is present in a concentration of about 5 mg/mL.

8. A composition according to claim 1, wherein said PTHrP analogue is present in a concentration from about 0.1 mg/mL to about 10.0 mg/mL.

9. A composition according to claim 1, wherein said PTHrP analogue is present in a concentration from about 1.0 mg/mL to about 2.0 mg/mL.

10. A composition according to claim 1, further comprising an effective amount of an isotonic agent.

11. A composition according to claim 10, wherein said isotonic agent is mannitol.

12. A composition according to claim 11, wherein said mannitol is present in a concentration from about 10 mg/mL to about 60 mg/mL.

13. A method of treating osteoporosis in a subject in need thereof comprising the administration to the subject a composition comprising a PTHrP analogue having the sequence [Glu^{22,25}, Leu^{23,28,31}, Aib²⁹, Lys^{26,30}]hPTHrP(1-34)NH₂ (SEQ ID NO.2), wherein the pH of the composition is in a range of about 3.5 to about 8.5;
provided that wherein said composition does not contain an effective amount of a pH buffer for maintaining the pH in a certain range.

14. The method according to claim 13, wherein said subject is administered the composition by single daily subcutaneous injection of an amount of said composition containing from about 70 to about 120 μg of [Glu^{22,25}, Leu^{23,28,31}, Aib²⁹, Lys26,30]hPTHrP(1-34)NH₂ (SEQ ID NO.2).

15. The method according to claim 13, wherein said composition further comprises phenol in a concentration from about 0.25 to about 7.0 mg/mL.

16. The method according to claim 13, wherein said composition further comprises mannitol in a concentration from about 10 mg/mL to about 60 mg/mL.

17. A composition suitable for administration to a subject comprising:
a) a PTHrP analogue having the sequence [Glu^{22,25}, Leu^{23,28,31}, Aib²⁹, Lys^{26,30}]hPTHrP(1-34)NH₂ (SEQ ID NO.2); and
b) an effective amount of a pH buffer to maintain the in a range of about 7.5 to about 8.5.

18. (canceled)

19. The composition according to claim 17, wherein said pH buffer is a phosphate buffer.

20. The composition according to claim 19, wherein said buffer is present in a concentration range of about 1 mM to about 100 mM.

21. The composition according to claim 17, further comprising an effective amount of phenol in a concentration from about 0.25 mg/mL to about 5 mg/mL.

22. The composition according to claim 17, further comprising an effective amount of mannitol in a concentration from about 10 mg/mL to about 60 mg/mL.
